(19) United States
(12) Patent Application Publication
(10) Pub. No.: US 2010/0130759 A1
(43) Pub. Date: May 27, 2010

(54) NOVEL FUNCTIONAL COMPOUNDS WITH AN ISOSORBIDE OR ISOSORBIDE ISOMER CORE, PRODUCTION PROCESS AND USES OF THESE COMPOUNDS

(75) Inventor: Jean-Philippe Gillet, Brignais (FR)

Correspondence Address: ARKEMA INC.;PATENT DEPARTMENT - 26TH FLOOR, 2000 MARKET STREET, PHILADELPHIA, PA, 19103-3222, US

(73) Assignee: Arkema France, Colombes (FR)

(21) Appl. No.: 12/597,518

(22) PCT Filed: Apr. 21, 2008
(86) PCT No.: PCT/FR08/50711

§ 371 Date: Oct. 26, 2009

(30) Foreign Application Priority Data
Apr. 27, 2007 (FR) 0754772

(51) Int. Cl. C07D 493/04

(52) U.S. Cl. 549/464

(57) ABSTRACT

The present invention relates to compounds of formula (I): R—(CH₂)₂—O-A-O—(CH₂)₂—R, in which A represents a divalent radical chosen from:

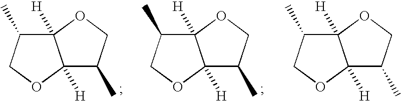

and R represents —CN or —CH₂NH₂. In order to prepare them, acrylonitrile is reacted, via a Michael reaction, with a compound of formula (II): HO-A-OH, in which A is as defined above, in order to obtain a compound of formula (I) in which R represents —CN, and that the hydrogenation of the latter is carried out in order to obtain the corresponding compound of formula (I) in which R represents —CH₂NH₂. Use is made of a compound of formula (I) in which R represents —CH₂NH₂ as a polar head in a surfactant, or as a monomer for a condensation polymerization, in particular in the manufacture of polyamides, or else as a crosslinking agents.

The present invention relates to novel functional compounds which comprise, as a core unit, an isosorbide unit or a unit of one of the two optical isomers of isosorbide, namely isomannide or isoidide. The present invention also relates to a process for preparing these novel functional compounds, and also to the applications thereof.

The isosorbide:

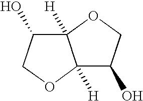

isomannide:

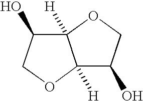

isoidide:

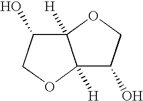

are natural substances obtained mainly from sugars derived from corn starch. The latter, via enzymatic reaction, gives glucose, which is reduced to sorbitol, the latter leading to isosorbide after a double dehydration:

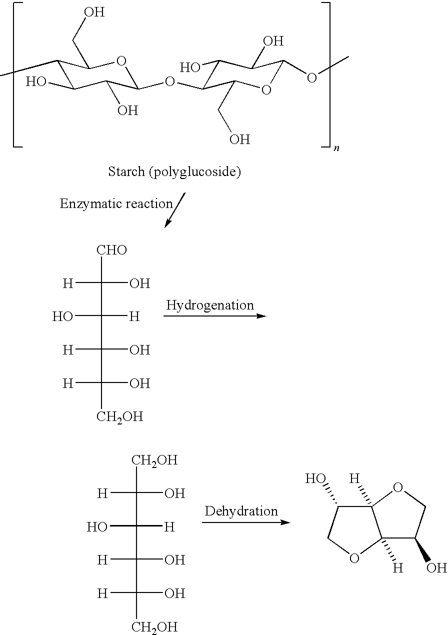

The optical isomers isomannide and isoidide are obtained in the same manner respectively from mannitol and from iditol. For more details on this chemistry, reference may be made, inter alfa, to the KIRK OTHMER encyclopedia, 4th edition, volume 23, pages 93 to 119.

At the present time, for the purpose of avoiding oil derivatives in the context of “green chemistry”, the performance chemicals industry is in search of novel compounds or monomers of natural, such as plant, origin that are therefore renewable, biodegradable, not very toxic and environmentally friendly. Furthermore, these novel compounds obtained from such raw materials should, preferably, be able to be obtained using clean energy and energy efficient processes.

Considering these requirements, the Applicant company has envisaged the synthesis of difunctional compounds bearing amine groups in particular from industrially accessible natural synthons that are isosorbide, isomannide and isoidide, and therefore the availability will increase in the next few years with the development of biorefineries.

The work of the Applicant company has then led to finding a process that makes it possible to convert the above synthons having an alcohol functional group in order to obtain novel compounds having nitrile and amine functional groups via a process that is simple and that can be easily scaled up to an industrial level.

This process relies on the principle of converting, in a first step, the alcohol functional groups to propionitrile ethers via a Michael reaction with acrylonitrile, then, in a second step, in converting the nitrile functional groups to primary amine functional groups via hydrogenation.

Thus, from heterocycloaliphatic bicyclic sugars, of plant, therefore renewable, origin and that are industrially available at low cost, the present invention provides a simplified access route—easy synthesis in two steps only, compatible with conventional industrial equipment—to original molecules, in particular molecules that are original due to their bicyclic core and their thermal stability:
Their bicyclic core may play, on the one hand, the role of a relatively large and hydrophilic polar head and, on the other hand, in the case where it could be used as a monomer, it may provide a certain rigidity in the materials.
Surprisingly, it has been observed that the thermal resistance of the novel compounds of the invention is excellent (being greater than 296° C.), which is far from being the case for plant-based products.

To the knowledge of the Applicant company, the compounds according to the present invention are novel, never being cited in the literature, apart from 2,5-bis-O-(3-aminopropyl)-1,4:3,6-dianhydro-D-glucitol, which is indeed cited by its CAS No. 6338-35-8, but which is not described in any document, any more than the method of obtaining it.

A diamine exists whose amine functional groups are directly borne by the isosorbide unit, namely 2,5-diamino-1,4:3,6-dianhydro-2,5-dideoxysorbitol or 2,5-diamino-1,4:3,6-dianhydro-2,5-dideoxy-D-glucitol (CAS 143396-56-9). However, as can be seen in the documents relating thereto, the synthesis is less direct, namely in three steps, and much more complicated since the yields range from 28 to 56%:

Synthesis (1994), 317-321;

International PCT application WO 9212978;

JACS (1956), 78, 3177-3182;

JCS (1950), 371-374;

Nature (1949), 164, 573-574.

Mention may also be made of the article in Bioorganic & Medicinal Chemistry Letters (2006), 16(3), 714-717, which relates to the molecular modeling of novel bis-cationic ligands with the lipid A site of a lipopolysaccharide. These are theoretical studies which aim to obtain molecules having a length of 14 angstroms in accordance with the receptor site. No description of the molecule or of its method of synthesis is found in this article.

The novel diamines of the present invention find an application as surfactants.

This is because, in the field of surfactants, the applications seek biodegradable and not very toxic products having, as raw materials, compounds of plant origin and therefore that are renewable. One of the means of responding to this problem is to use condensation chemistry between, on the one hand, a lipophilic fatty chain—originating from fatty acids—corresponding to these criteria and a hydrophilic amino synthon joined together by a cleavable chemical functional group, such as the amide functional group. Generally the polyamines used: diethylenetriamine (DETA), triethylenetetramine (TETA), etc., are of oil origin and have an impact on the environment. The present invention therefore makes it possible to readily obtain a diamine based on a plant raw material responding to the criteria of biodegradability and of low toxicity.

The novel diamines of the invention also have an application, in the field of materials, as monomers in condensation polymerization reactions, for example for the manufacture of polyamides, and also as crosslinking agents. Their very good thermal stability and their plant origin constitute criteria of choice in such applications.

One subject of the present invention is therefore firstly compounds of formula (I):

R— (CH₂)₂—O-A-O—(CH₂)₂—R (I)

in which:

A represents a divalent radical chosen from:

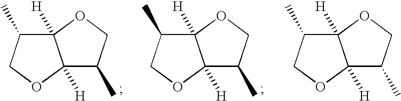

the two free bonds in each of the three formulae above constituting the points of attachment of the group A to the oxygen atoms in the formula (I),
and

R represents —CN or —CH₂NH₂.

Mention is particularly made of the compounds of the present invention represented by the formulae:

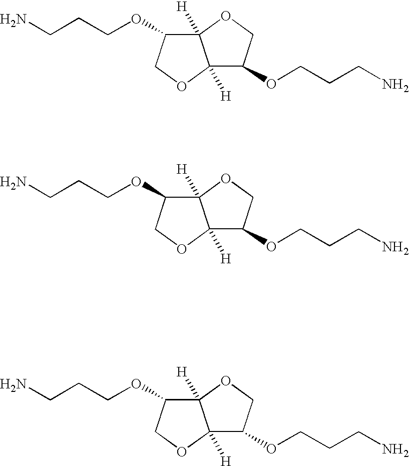

Another subject of the present invention is a process for manufacturing a compound of formula (I) as defined above, characterized by the fact that acrylonitrile is reacted, via Michael reaction, with a compound of formula (II):

HO-A-OH (II)

in which A is as defined in claim 1, in order to obtain a compound of formula (I) in which R represents —CN, and that the hydrogenation of the latter is carried out in order to obtain the corresponding compound of formula (I) in which R represents —CH₂NH₂.

### First Step: Michael Reaction

Acrylonitrile is reacted with the compound of formula (II), especially with an acrylonitrile/(compound (II)×2) molar ratio of 1 to 2, preferably of 1 to 1.5.

This step is generally carried out at a temperature of 20° C. to 100° C., preferably 40° C. to 80° C.

Equally, this step is advantageously carried out in presence of at least one basic or non-basic catalyst, used in particular in an amount of 0.05% to 5% by weight, preferably 0.1 to 3% by weight, relative to the compound of formula (II).

The basic catalyst(s) may be chosen from:
alkali metal hydroxides, such as Li, Na, K, Rb or Cs hydroxide;
alkaline-earth metal hydroxides, such as Mg, Ca, Sr or Ba hydroxide;
Li, Na, K, Rb or Cs carbonates;
alkali or alkaline-earth metal alcoholates, such as sodium methylate, sodium ethylate and potassium tert-butylate; and
basic heterogeneous catalysts, such as basic resins, zeolites, hydrotalcite and magnesium oxide.

Among the other non-basic catalysts (including those having a less pronounced basic character), mention may be made of K fluoride and Cs fluoride, which are pure or supported, for example on alumina.

In accordance with a first embodiment, use is made of the compound of formula (II) alone in the molten state.

In accordance with a second embodiment, use is made of the compound of formula (II) in solution in a solvent such as tert-butanol in the case of a low-temperature Michael reaction, aromatic hydrocarbons, such as toluene, and polar aprotic solvents, such as acetonitrile.

Finally, this Michael reaction is generally carried out at atmospheric pressure, but there is no drawback to working under a slight pressure owing to the boiling point of acrylonitrile, which is 77° C.

This first step can be described in greater detail as follows:

Isosorbide or its isomannide or isoidide isomer may be used alone in the molten state (MP: 60-63° C.) in the case of isosorbide or in solution in a solvent, such as test-butanol for the low temperatures, that is to say at a temperature below the melting point of the raw material, aromatic hydrocarbons (for example toluene), polar aprotic solvents (for example acetonitrile). A basic catalyst is introduced, which is generally an alkali or alkaline-earth metal hydroxide, such as those indicated above, but it is also possible to use alkali or alkaline-earth metal alcoholates and also basic heterogeneous catalysts, alkali metal carbonates or potassium and cesium fluorides, examples of which are cited above. The mixture is heated between 40° C. and 80° C., then the acrylonitrile is introduced. The reaction is continued until the conversion of the alcohol functional groups to ethers. The reaction product may be used crude, but it is also possible to purify it by high vacuum distillation. It is also possible to neutralize the catalyst with an acid.

### Second Step: Hydrogenation

Advantageously, the hydrogenation is carried out in the presence of ammonia, with an NH₃/CN molar ratio generally of 0.2 to 2.5, preferably of 0.5 to 1.5.

This hydrogenation is carried out under the following advantageous conditions:
at a temperature generally of 40° C. to 180° C., preferably 50° C. to 130° C.;
in a pressurized reactor at a total pressure of 5×10⁵ Pa to 1.50×10⁷ Pa (5 bar to 150 bar), preferably 2×10⁶ Pa to 8×10⁶ Pa (20 bar to 80 bar);
in the presence of at least one hydrogenation catalyst, in an amount, especially, of 0.1 to 20% by weight, preferably 0.5% to 10% by weight, relative to the compound of formula (I) in which R represents —CN, the hydrogenation catalyst(s) being advantageously chosen from Raney nickel, Raney cobalt, palladium and rhodium, the latter two catalysts possibly being supported on charcoal or alumina.

In accordance with a first embodiment, the hydrogenation is carried out without solvent.

In accordance with a second embodiment, the hydrogenation is carried out in a solvent medium, the solvent(s) being compatible with the hydrogenation reaction and being chosen, in particular, from water and linear or branched C₁ to C₅ light alcohols.

The second step can be described more particularly and in greater detail as follows:

A pressurized reactor is used. It is possible to operate without solvent or in a solvent medium, solvents that can be used by way of example being cited above. The reactor is charged with the ether dinitrile and the catalyst. The catalyst is chosen from the conventional catalysts for hydrogenation of nitriles, such as those cited above. For cost reasons, Raney nickel and Raney cobalt are preferred. The reactor is sealed and then ammonia is introduced. The reaction medium is stirred and brought to a temperature between 50° C. and 150° C. Next the hydrogen is introduced. The reaction starts and is continued until the complete conversion of the nitrile functional groups to amine functional groups. The amount of ammonia is judiciously chosen so as to minimize secondary amine formation. At the end of the reaction, the catalyst is filtered, and may be recycled. The solvent is evaporated where necessary. The diamine is possibly purified by high vacuum distillation or recrystallization of its hydrochloride form.

It is also possible to proceed according to a variant of this process which consists in charging the reactor with a solvent, the catalyst, ammonia, hydrogen and continuously introducing ether dinitrile and hydrogen in order to maintain the pressure of the reaction. The purpose, here too, is to promote the formation of primary amines at the expense of secondary amines.

The present invention also relates to the use of a compound of formula (I) in which R represents —CH₂NH₂ as a polar head in a surfactant, or as a monomer (comonomer) for a condensation polymerization, in particular in the manufacture of polyamides, or else as a crosslinking agent, and also to the use of a compound of formula (I) in which R represents —CN as a synthesis intermediate in the preparation of compounds of formula (I) in which R represents —CH₂NH₂.

The following examples illustrate the present invention without however limiting the scope thereof. In these examples, the percentages are by weight unless otherwise indicated.

### EXAMPLE 1

### Synthesis of 2,5-bis-O-(propionitrile)-1,4:3,6-dianhydro-D-sorbitol

A 500 cm³ jacketed glass reactor, equipped with a stirrer, a dropping funnel, and a condenser was charged with 100 g (0.68M) of isosorbide and 0.5 g, i.e. 5000 ppm, of sodium hydroxide pearls. The reaction medium was brought to 70-75° C. until the sodium hydroxide had completely dissolved and the isosorbide had melted. Then 90.1 g (1.7M), i.e. 25% excess of acrylonitrile relative to the alcohol functional groups, were added slowly. At the end of the reaction, the excess acrylonitrile was evaporated and the crude reaction product was recovered. The yield of the expected product was 90%.

### Analytical Characterization of the Product

### 2,5-bis-O-(Propionitrile)-1,4:3,6-dianhydro-D-sorbitol

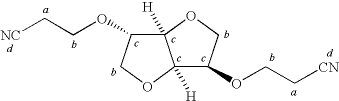

¹³C NMR in CDCl₃

δa=18.31 ppm
δb=63.45 ppm; 64.51 ppm; 70.02 ppm and 72.48 ppm
δc=79.65 ppm; 79.97 ppm; 83.69 ppm and 85.35 ppm
δd=117.48 ppm and 117.55 ppm.

### EXAMPLE 2

### Synthesis of 2,5-bis-O-(3-aminopropyl)-1,4:3,6-dianhydro-D-sorbitol

Introduced into a 500 cm³ autoclave were 200 g of the crude reaction product obtained previously, with 10 g. of wet Raney nickel. The autoclave was sealed. Then 15 g of ammonia were introduced at ambient temperature (i.e. an NH₃/CN molar ratio of 0.55). The reaction medium was brought, with stirring, to 130° C. The total pressure was brought to 2.5×10⁶ Pa (25 bar) by introduction of hydrogen. The pressure and the temperature were maintained at these values throughout the entire reaction. When the reaction was terminated, the crude reaction product was recovered by filtration in order to recover the catalyst which can be recycled. The yield was 85%. The diamine can be obtained pure by high pressure distillation (BP: 165-175° C. under 133.322 Pa (1 mmHg)).

### Analytical Characterization of the Product

### 2,5-bis-O-(3-Aminopropyl)-1,4:3,6-dianhydro-D-sorbitol

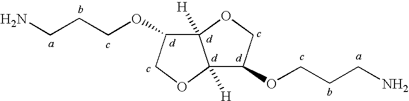

Confirmation of the mass by GC-MS coupling.

¹³C NMR in CD₃OD

δa=40.42 ppm and 40.38 ppm
δb=34.29 ppm and 34.11 ppm
δc=69.16 ppm; 69.97 ppm; 71.69 ppm and 74.69 ppm
δd=81.94 ppm; 82.09 ppm; 86.08 ppm and 87.85 ppm.

### EXAMPLE 3

### Synthesis of 2,5-bis-O-(propionitrile)-1,4:3,6-dianhydro-D-sorbitol

A 500 cm³ predried glass reactor equipped with effective mechanical stirring, heating, a condenser, a dropping funnel and a nitrogen inerting system was charged with 46.2 g (316 mmol) of isosorbide with 98.2 g of tert-butanol and 1 g of lithium hydroxide. The reaction medium was brought to 60° C., then 50.3 g (949 mmol) of acrylonitrile was poured in over a duration of 1 h 30 min. The reactions continued for a total duration of 8 h.

The catalyst was neutralized with an acid solution, then the residual tert-butanol and acrylonitrile were evaporated under reduced pressure. Thus 80.8 g of crude product containing 90% of dinitrile (HPLC assay) were obtained. The conversion of isosorbide was 95% and the yield was 91%.

### Analytical Characterization of the Product

### Cf. Example 1

### EXAMPLE 4

### Synthesis of 2,5-bis-O-(3-aminopropyl)-1,4:3,6-dianhydro-D-sorbitol

A 300 cm³ autoclave was charged with 100 g of water, 12 g of (50%) wet Raney nickel and 2.6 g of ammonia. The reactor was pressurized with hydrogen up to a total pressure of 6×10⁶ Pa (60 bar) for a temperature of 60° C. A solution of 34.5 g of the crude reaction product in 30 g of water was introduced continuously. The introduction was carried out over 3 h 15 min and the pressure and the temperature were maintained at the aforementioned values. At the end of the reaction, the medium was cooled, the catalyst filtered, and the solvent evaporated. Thus 28.5 g of crude product containing 79% of diamine were obtained. The yield was 70% of diamine.

### Analytical Characterization of the Product

### Cf. Example 2

1. A compound of formula (I):
R—(CH₂)₂—O-A-O—(CH₂)₂—R (I)
in which:
A represents a divalent radical chosen from: 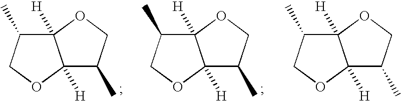
and
R represents —CN or —CH₂NH₂.

2. The compound as claimed in claim 1, which is represented by the formula:

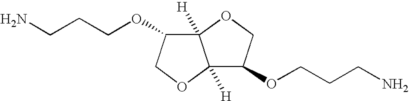

3. The compound as claimed in claim 1, which is represented by the formula:

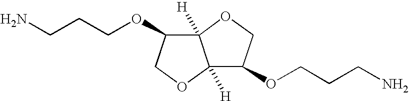

4. The compound as claimed in claim 1, which is represented by the formula:

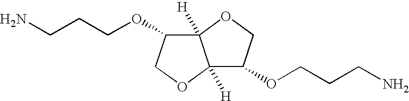

5. A process for manufacturing a compound of formula (I) according to claim 1, comprising the steps of:
a) reacting acrylonitrile, via the Michael reaction, with a compound of formula (II):
HO-A-OH (II)
in which A is as defined in claim 1, in order to obtain a compound of formula (I) in which R represents —CN, and
b) converting the nitrile functional groups to primary amine functional groups via hydrogenation of the compound of formula (I) in which R represents —CN in order to obtain the corresponding compound of formula (I) in which R represents —CH₂NH₂.

6. The process as claimed in claim 5, wherein said acrylonitrile is reacted with the compound of formula (II) with an acrylonitrile/(compound (II)×2) molar ratio of 1 to 2.

7. The process as claimed in claim 5, wherein the acrylonitrile is reacted with the compound of formula (II) at a temperature of 20° C. to 100° C.

8. The process as claimed in claim 5, wherein the acrylonitrile is reacted with the compound of formula (II) in the presence of at least one basic or non-basic catalyst, used in an amount of 0.05 to 5% by weight, relative to the compound of formula (II).

9. The process as claimed in claim 8, wherein the basic catalyst(s) is(are) chosen from:
alkali metal hydroxides, such as Li, Na, K, Rb or Cs hydroxide;
alkaline-earth metal hydroxides, such as Mg, Ca, Sr or Ba hydroxide;
Li, Na, K, Rb or Cs carbonates;
alkali or alkaline-earth metal alcoholates, such as sodium methylate, sodium ethylate and potassium text-butylate; and
basic heterogeneous catalysts, such as basic resins, zeolites, hydrotalcite and magnesium oxide,
and the non-basic catalyst(s) is(are) chosen from K fluoride and Cs fluoride, pure or supported.

10. The process as claimed in claim 5, wherein the compound of formula (II) is used alone in the molten state.

11. The process as claimed in claim 5, wherein use is made of the compound of formula (II) in solution in a solvent in the case of a low-temperature Michael reaction, aromatic hydrocarbons, and polar aprotic solvents.

12. The process as claimed in claim 5, wherein the Michael reaction is carried out at atmospheric pressure or under a slight pressure.

13. The process as claimed in claim 5, wherein the hydrogenation is carried out in the presence of ammonia, with an NH₃/CN molar ratio of 0.2 to 2.5.

14. The process as claimed in claim 5, wherein the hydrogenation is carried out at a temperature of 40° C. to 180° C.

15. The process as claimed in claim 5, wherein the hydrogenation is carried out in a pressurized reactor at a total pressure of 5×10⁵ Pa to 1.5×10⁷ Pa (5 bar to 150 bar).

16. The process as claimed in claim 5, wherein the reaction is carried out in the presence of at least one hydrogenation catalyst, in an amount of 0.1 to 20% by weight relative to the compound of formula (I) in which R represents —CN.

17. The process as claimed in claim 16, wherein the hydrogenation catalyst(s) is(are) chosen from Raney nickel, Raney cobalt, palladium and rhodium, the latter two catalysts optionally being supported on charcoal or alumina.

18. The process as claimed in claim 5, wherein the hydrogenation is carried out without solvent.

19. The process as claimed in claim 5, wherein the hydrogenation is carried out in a solvent medium, the solvent(s) being compatible with the hydrogenation reaction and being chosen from water and linear or branched C₁ to C₅ light alcohols.

20. The compound of formula (I) in which R represents —CH₂NH₂ comprising a polar head in a surfactant, a monomer for a condensation polymerization, or else as a crosslinking agent.

21. The compound of formula (I) in which R represents —CN comprising a synthesis intermediate in the preparation of compounds of formula (I) in which R represents —CH₂NH₂.
